(19) United States
(12) Patent Application Publication
(10) Pub. No.: US 2023/0070396 A1
(43) Pub. Date: Mar. 9, 2023

(54) NOVEL STRUCTURIZING OIL, METHOD OF PRODUCTION, AND USES IN MARGARINE AND ICE CREAM

(71) Applicant: PALSGAARD A/S, Juelsminde (DK)

(72) Inventors: Kim CHRISTIANSEN, Juelsminde (DK); Lars PREUSS, Juelsminde (DK); Claus Hviid CHRISTENSEN, Juelsminde (DK); Allan Torben BECH, Juelsminde (DK)

(21) Appl. No.: 17/800,258

(22) PCT Filed: Feb. 19, 2021
(86) PCT No.: PCT/EP2021/054174

§ 371 Date: Aug. 17, 2022

(30) Foreign Application Priority Data
Feb. 20, 2020 (EP) 20158532.0

(51) Int. Cl. A23D 7/04; A23D 7/015; A23D 7/01; A23G 9/20; A23D 7/005; A21D 2/16; C12P 7/6436; C12N 9/20

(52) U.S. Cl. CPC A23D 7/04; A23D 7/015; A23D 7/013; A23G 9/20; A23D 7/0056; A21D 2/165; C12P 7/6436; C12N 9/20

(57) ABSTRACT

The present invention pertains to the production of a novel structurizing oil by enzymatic interesterification which novel structurizing oil is particularly well suited as oil ingredient in e.g. margarines or ice creams.

### FIELD OF THE INVENTION

The present invention pertains to the production of a novel structurizing oil which is particularly well suited as oil ingredient in e.g. margarines or ice creams.

### BACKGROUND

Food products such as margarines and ice creams contain a considerable amount of fat which contributes to the sensory characteristics of the products. Such food products may contain a structurizing oil.

A structurizing oil can be used in the production of margarines and spreads where it contributes to the control of the crystallization and structurizing of the fat to give a better texture and/or melting profile. Structurizing oil is also helpful in other applications where the control of the behaviour of the triglycerides is important, i.e. in ice-cream or bakery goods.

### SUMMARY OF THE INVENTION

The present inventors have found that a new structurizing oil with advantageous properties can be produced by enzymatic interesterification of one or more vegetable oils in the presence of water and subsequent removal of free fatty acids.

Thus, an aspect of the invention pertains to a method of producing a structurizing oil comprising:
a) subjecting a mixture comprising a first vegetable oil, water, and enzyme having lipase activity to interesterification,
b) subjecting the interesterified mixture of step a) to:
   i) reduction of the lipase activity,
   ii) reduction of the content of free fatty acids (ffa) selectively relative to the partial glycerides,
   iii) optionally, reduction of the water content,
wherein steps i), ii), and iii) may be performed in any order, thereby providing a ffa-reduced mixture,
c) optionally, mixing the ffa-reduced mixture of step b) with a second vegetable oil to obtain an oil blend,
wherein the ffa-reduced mixture of step b) and/or the oil blend of step c) is recovered as the structurizing oil.

The inventors have discovered that the oil of the present invention acts as a structurizing agent in fat-containing food matrices such as e.g. margarines and frozen, aerated confections and provides a reduced droplet size of water or fat and an improved stability of the resulting product.

Another aspect of the invention pertains to a structurizing oil, preferably obtainable by the method described herein, containing
triglyceride in an amount in the range of 50-95% w/w,
diglyceride in an amount in the range of 2-40% w/w,
monoglyceride in an amount in the range of 0.2-6% w/w,
free fatty acids in an amount of at most 4% w/w,
glycerol in an amount of at most 1% w/w.

Further aspects of the invention pertain to the use of the structurizing oil as oil source for production of fat-containing food products such as e.g. margarine-type products or frozen, aerated confections.

### BRIEF DESCRIPTION OF THE FIGURES

FIG. 1 shows the distribution of fatty acids in triglycerides prior to and during interesterification.

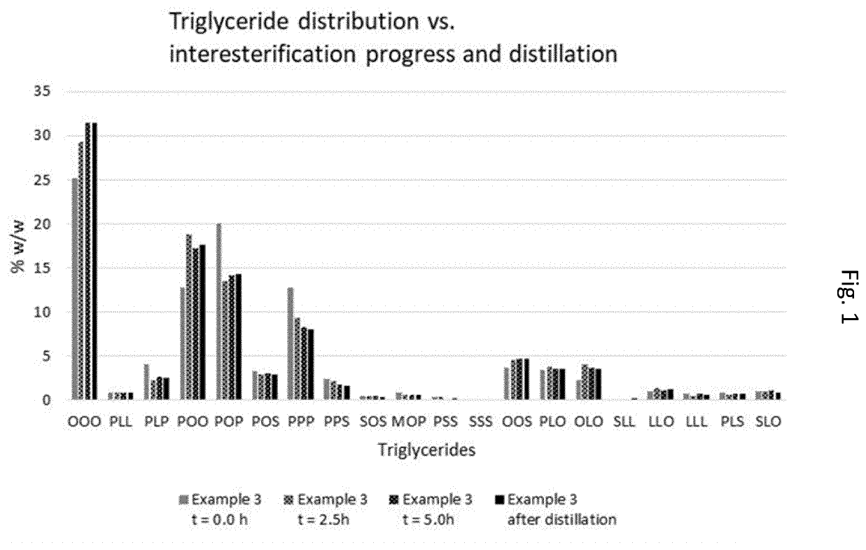

FIG. 2 shows the results of a squeeze test applied to the margarine samples of Example 4.

FIG. 3 shows the results of a squeeze test applied to the margarine samples of Example 5.

### DETAILED DESCRIPTION

An aspect of the invention pertains to a method of producing a structurizing oil comprising:
a) subjecting a mixture comprising a first vegetable oil, water, and enzyme having lipase activity to interesterification,
b) subjecting the interesterified mixture of step a) to:
   i) reduction of the lipase activity,
   ii) reduction of the content of free fatty acids (ffa) selectively relative to the partial glycerides,
   iii) optionally, reduction of the water content,
wherein steps i), ii), and iii) may be performed in any order, thereby providing a ffa-reduced mixture,
c) optionally, mixing the ffa-reduced mixture of step b) with a second vegetable oil to obtain an oil blend,
wherein the ffa-reduced mixture of step b) and/or the oil blend of step c) is recovered as the structurizing oil.

An advantage of the present invention is that it makes it possible to produce the present structurizing oils under mild conditions which allows for structurizing oils having low peroxide values and low contents of glycidol equivalents. This is even more pronounced for unsaturated structurizing oils having e.g. an iodine value of at least 40 g/100 g.

Step a) involves subjecting a mixture comprising a first vegetable oil, water, and enzyme having lipase activity to interesterification. The interesterification is preferably catalysed by the enzyme.

In the context of the present invention the term “vegetable oil” pertains to oils derived from vegetable sources and encompasses also modified vegetable oils such as e.g. refined vegetable oils, fractionated vegetable oils, and hydrogenated vegetable oils.

In the context of the present invention the term “oil” includes both oils that are liquid at room temperature and oils that are solid or semi-solid at room temperature. The terms “oil” and “fat” are used interchangeably herein.

In the context of the present invention the term “first vegetable oil” is used to describe the total amount of vegetable oil of the mixture and may e.g. contain oil of a single or several vegetable oil sources.

In the context of the present invention the term “second vegetable oil” pertains to the total amount of vegetable oil that optionally is mixed with the interesterified mixture of obtained from step b). The second vegetable oil may have the same composition as the first vegetable oil, or it may have a different composition than the first vegetable oil and it may comprise a single or several vegetable oil sources.

The term mixture is used to describe the composition in which the interesterification takes place. If the enzyme has been immobilised the solid phase holding the enzyme is as such not considered part of the mixture but the enzyme is.

In the context of the present invention the term “enzyme having lipase activity” pertains to lipases or enzymes having lipase activity in addition to another activity. Lipases (EC Number 3.1.1.3) are one of the most commonly used classes of enzymes in biocatalysis. They have been used on a variety of substrates and show very broad substrate specificity. Lipases catalyze the hydrolysis of triacylglycerols to diacylglycerol, monoacylglycerol, glycerol and free fatty acids. The reaction reverses under anhydrous conditions and the enzyme is able to synthesize new molecules by esterification, alcoholysis and/or transesterification. All reactions can be performed with high regio- and enantioselectivity under mild reaction conditions.

Esterases and lipases are subclasses of the hydrolase superfamily of enzymes. Lipases have been historically differentiated from esterases by their ability to hydrolyze glycerol esters of long-chain fatty acids, as well as being enzymatically activated at the lipid—water interface. The bacterial lipases/esterases encompass a large superfamily of enzymes that has been classified into eight major families (Reetz, M. T. (2002). Lipases as practical biocatalysts. Curr. Opin. Chem. Biol.6, 145-150).

Lipase activity is measured in the unit KLU (K=Kilo; LU=Lipase units) according to Food Chemical Codex (FCC) III/general tests and Apparatus/1981/493.

The enzyme having lipase activity preferably has a lipase activity of at least 1 KLU/g.

In the context of the present invention the term “structurizing oil” pertains to the oil product of the present invention which comprises or even consists of the interesterified, ffa-reduced first vegetable oil, and optionally a second vegetable oil. The inventors have found that this oil product has structurizing effects when used in e.g. margarines and ice creams.

In some preferred embodiments of the present invention the mixture comprises the first vegetable oil in an amount in the range of 10-99.9% w/w, more preferably 30-99.8% w/w, even more preferably 50-99.7% w/w, and most preferably 60-99.7% w/w.

In some preferred embodiments of the present invention the first vegetable oil has an iodine value of at least 20, more preferably at least 40, even more preferably at least 50, and most preferably at least 60.

The iodine value is measured according to Analysis F and is expressed as grams of iodine/100 g.

Preferably, the first vegetable oil has an iodine value in the range of 20-100, even more preferably in the range of 40-90, and most preferably in the range of 50-90.

In other preferred embodiments of the present invention the first vegetable oil has an iodine value in the range of 30-130, more preferably in the range of 40-120, even more preferably in the range of 40-110, and most preferably in the range of 40-100.

In further preferred embodiments of the present invention the first vegetable oil has an iodine value in the range of 30-120, more preferably in the range of 50-110, even more preferably in the range of 50-100, and most preferably in the range of 50-95.

It is often preferred that the first vegetable oil has a least some unsaturated fatty acids as this gives rise to a lower melting point and hence a lower temperature during interesterification and better stability of the enzyme.

In some preferred embodiments of the present invention the first vegetable oil has a peroxide value of at most 10 meq/kg, more preferably at most 5 meq/kg, even more preferably at most 3 meq/kg, and most preferably at most 2 meq/kg.

In some preferred embodiments of the present invention the first vegetable oil has a content of glycidol equivalents of at most 10 ppm, more preferably at most 5 ppm, even more preferably at most 1 ppm, and most preferably at most 0.5 ppm.

In some preferred embodiments of the present invention the first vegetable oil comprises a saturated vegetable oil and an unsaturated vegetable oil.

If the first vegetable oil contains several vegetable oils these may be added individually or may be premixed and added as the premix.

In the context of the present invention a “saturated vegetable oil” has an iodine value of less than 50 and an “unsaturated vegetable oil” has an iodine value of at least 50.

The saturated vegetable oil is preferably selected from the group consisting of palm stearin, palm kernel oil, shea stearin, coconut oil, hydrogenations of vegetable oil, fractions thereof, and/or a blend thereof.

The saturated vegetable oil is preferably selected form the group consisting of palm stearin, palm kernel oil, shea stearin, a hydrogenated vegetable oil or a combination thereof.

In some preferred embodiments of the present invention the first vegetable oil is a blend of different vegetable oils and/or oil fractions.

Preferably, the first vegetable oil comprises the saturated vegetable oil in an amount in the range of 1-80% w/w, more preferably 10-70% w/w, even more preferably 20-60% w/w, and most preferably 20-50% w/w.

Preferably, the first vegetable oil comprises the saturated vegetable oil in an amount in the range of 1-50% w/w, more preferably 2-40% w/w, even more preferably 4-30% w/w, and most preferably 4-25% w/w.

The saturated vegetable oil preferably has an iodine value in the range of 0-49, more preferably in the range of 10-45, even more preferably in the range of 14-42, and most preferably in the range of 20-40.

The first vegetable oil preferably comprises the unsaturated vegetable oil in an amount in the range of 20-99% w/w, more preferably 30-80% w/w, even more preferably 40-80% w/w, and most preferably 40-70% w/w.

In other preferred embodiments of the present invention the first vegetable oil comprises the unsaturated vegetable oil in an amount in the range of 20-100% w/w, more preferably 30-100% w/w, even more preferably 40-100% w/w, and most preferably 50-100% w/w.

The unsaturated vegetable oil preferably has an iodine value in the range of 50-130, more preferably in the range of 70-120, even more preferably in the range of 80-110, and most preferably in the range of 85-100.

In some preferred embodiments of the present invention the unsaturated vegetable oil is selected from the group consisting of Rapeseed oil, Sunflower oil, High oleic rapeseed oil, High oleic sunflower oil, Almond oil, avocado oil, Camelina oil, Corn oil, Cottonseed oil, Linseed oil, Olive oil, Palm oil, Palm olein, Peanut oil, Hazelnut oil, Mustard seed oil, Pumpkin seed oil, Rice bran oil, Safflower oil, Sesame oil, Soybean oil, Shea oil, Shea olein, Walnut oil, and a combination thereof.

More preferably the unsaturated vegetable oil is selected from the group consisting of high oleic sunflower oil, high oleic rapeseed oil, palm olein, shea olein, and a combination thereof.

In some preferred embodiments of the present invention the mixture comprises a total amount of triglyceride in the range of 50-99.9% w/w, more preferably 60-99% w/w, even more preferably 70-97% w/w, and most preferably 75-95% w/w.

In some preferred embodiments of the present invention the mixture comprises a total amount of diglyceride in the range of 0-35% w/w, more preferably 1-25% w/w, even more preferably 2-15% w/w, and most preferably 2-10% w/w.

In some preferred embodiments of the present invention the mixture comprises a total amount of monoglyceride in the range of 0-15% w/w, more preferably 0-12% w/w, even more preferably 0-10% w/w, and most preferably 0-8% w/w.

In some preferred embodiments of the present invention the mixture comprises water in an amount in the range of 0.001-5% w/w, more preferably 0.01-2% w/w, even more preferably 0.05-1% w/w, and most preferably 0.1-0.5% w/w.

In some preferred embodiments of the present invention the mixture comprises the lipase in an amount of 0.01-10% w/w relative to the weight of the oil, more preferably 0.05-7% w/w, even more preferred 0.1-5% w/w, and most preferred 0.5-4% w/w relative to the weight of the oil.

In some preferred embodiments of the present invention the mixture comprises the enzyme in an amount sufficient to provide a lipase activity relative to the amount of first vegetable oil in the range of 0.1-5 KLU per gram of the first vegetable oil, more preferred 0.5-3 KLU per gram of the first vegetable oil, even more preferred 0.5-1 KLU per gram of the first vegetable oil.

It is particularly preferred that the enzyme comprises or even consists of a lipase.

In some preferred embodiments of the present invention the enzyme comprises or even consists of a 1,3-specific lipase.

In other preferred embodiments of the present invention the enzyme comprises or even consists of a lipase that is not 1,3-specific.

A preferred example of a useful enzyme is Lipozyme® TL from Novozymes, which is a 1,3-specific lipase originating from Thermomyces lanuginosus with declared activity 100 KLU/g.

The enzyme is typically present in free or immobilised form. It is most preferred that the enzyme is present in free form.

The enzyme is preferably added in solved, preferably hydrated, form or in dried form.

The enzyme may e.g. be provided in a solution comprising water, glycerol, and/or sorbitol and the mixture may therefore contain minor amounts of these components.

The interesterification of the present invention is catalysed by the enzyme having lipase activity. The interesterification preferably involves heating the mixture to a temperature in the range of 20 to 80 degrees C., more preferably in the range of 25 to 70 degrees C., and most preferably in the range of 30 to 60 degrees C.

The mixture preferably has a pH of 3-9, more preferably 4-8, even more preferably 5-8, and most preferably 6-8.

The pH of the mixture is measured in a 10% dispersion of the mixture in distilled water at 20 degrees C.

The progress in the interesterification typically is followed by analysis of the composition by GC and the triglyceride distribution by GC-FID (DGF C-VI 14). The interesterification typically takes place until the progress slows down and almost no changes are observed. This typically happens after 3-6 hours depending of the reaction parameters such as the temperature and enzyme activity. Step b) involves subjecting the interesterified mixture of step a) to:
i) reduction of the lipase activity,
ii) reduction of the content of free fatty acids (ffa) selectively relative to the partial glycerides,
iii) optionally, reduction of the water content,
wherein steps i), ii), and iii) may be performed in any order, thereby providing a ffa-reduced mixture,

Step b) preferably performed in one of the following the sequences:
i) followed by ii),
i) followed by ii) followed by iii),
i) followed by iii) followed by ii),
ii) followed by i),
ii) followed by i) followed by iii),
ii) followed by iii) followed by i),
iii) followed by i) followed by ii), or
iii) followed by ii) followed by i).

The reduction or even removal of enzyme having lipase activity may be accomplished in different ways, e.g. by inactivation and optionally removal of the inactivated enzyme or by removal of the solid phase holding the active enzyme.

In some preferred embodiments of the present invention step b.i) involves inactivating the enzyme, preferably heating to a temperature in the range of 80 to 250 degrees C., and more preferably 85 -120 degrees C., and most preferably 90-100 degrees C. Preferably the heat-treatment is as short as possible to avoid damaging the oil.

Preferably, step b.i) furthermore involves removing the inactivated enzyme, e.g. by filtration or centrifugation, preferably by filtration.

In other preferred embodiments of the present invention step b.i) involves removing the solid phase holding the enzyme from the interesterified mixture.

Step b.ii) involves a reduction of the content of ffa of the interesterified mixture.

The reduction of the ffa content is preferably selective meaning that the reduction in the concentration of ffa (relative to the concentration of ffa in the interesterified mixture) is larger than then reduction in partial glycerides (relative to the concentration of partial glycerides in the interesterified mixture).

In some preferred embodiments of the present invention step b.ii) involves or even consists of distillation and/or extraction, most preferably distillation. In some preferred embodiments of the present invention the extraction is performed by contacting the interesterified mixture with glycerol on the presence of an alkalizing agent as described in WO2012035020 A1.

In some preferred embodiments of the present invention the distillation of step b.ii) involves or even consists of thin layer distillation or short path distillation.

In some preferred embodiments of the present invention the distillation of step b.ii) involves or even consists of distillation under vacuum.

When used, the distillation of step b.ii) preferably employs a distillation temperature in the range of 140 to 220 degrees C., and most preferably in the range of 150 to 180 degrees C.

When used, the distillation of step b.ii) preferably employs a distillation pressure in the range of 0.1 Pa to 200 Pa, and most preferably in the range of 1 Pa to 100 Pa.

Preferably, the content of ffa of the interesterified mixture is reduced to at most 5% w/w, more preferably at most 3% w/w, even more preferably at most 2% w/w, and most preferably at most 1% w/w.

Alternatively, but also preferred, the content of ffa of the interesterified mixture may be reduced to at most 0.8% w/w, more preferably at most 0.5% w/w, even more preferably at most 0.3% w/w, and most preferably at most 0.1% w/w.

The method may furthermore comprise b.iii) which involves reducing the water content.

Preferably, the water content of the interesterified mixture is reduced to at most 1% w/w, more preferably at most 0.5% w/w, even more preferably at most 0.1% w/w, and most preferably at most 0.01% w/w.

Step c) is optional and involves mixing the ffa-reduced mixture of step b) with a second vegetable oil to obtain an oil blend.

Step c) is optional in the sense that some preferred embodiments of the present invention comprise step c) while other preferred embodiments of the present invention do not comprise step c). If step c) is included the ffa-reduced mixture is mixed with a second vegetable oil to form the structurizing oil. If step c) is not included the ffa-reduced mixture is preferably used as the structurizing oil.

Thus, in some preferred embodiments of the present invention the method comprises step c).

In some preferred embodiments of the present invention, the oil blend obtained from step c) comprises the ffa-reduced mixture of step b) in an amount of at least 2% w/w, more preferably at least 5% w/w, and most preferably at least 15% w/w.

In other preferred embodiments of the present invention, the oil blend obtained from step c) comprises the ffa-reduced mixture of step b) in an amount of at least 40% w/w, more preferably at least 70% w/w, and most preferably at least 90% w/w.

In further preferred embodiments of the present invention the oil blend obtained from step c) comprises the ffa-reduced mixture of step b) in an amount of 2-40% w/w, more preferably 5-30% w/w, and most preferably 10-25% w/w.

In other preferred embodiments of the present invention, the oil blend obtained from step c) comprises the ffa-reduced mixture of step b) in an amount of 2-25% w/w, more preferably 2-15% w/w, and most preferably 2-6% w/w.

The second vegetable oil preferably makes up the part of the oil blend that is not provided by the ffa-reduced mixture.

In some preferred embodiments of the present invention, the oil blend obtained from step c) comprises the second vegetable oil in an amount of at least 0.1% w/w, more preferably at least 1% w/w, and most preferably at least 10% w/w.

In other preferred embodiments of the present invention, the oil blend obtained from step c) comprises the second vegetable oil in an amount of 72-98% w/w, more preferably 82-98% w/w, and most preferably 92-98% w/w.

In further preferred embodiments of the present invention, the oil blend obtained from step c) comprises the second vegetable oil in an amount of 55-98% w/w, more preferably 65-95% w/w, and most preferably 70-90% w/w.

In some preferred embodiments of the present invention the second vegetable oil has an iodine value of at least 20, more preferably at least 40, even more preferably at least 50, and most preferably at least 60.

The iodine value is measured according to Analysis F and is expressed as grams of iodine/100 g.

Preferably, the second vegetable oil has an iodine value in range of 20-100, even more preferably in the range of 40-90, and most preferably in the range of 50-90.

In other preferred embodiments of the present invention the second vegetable oil has an iodine value in the range of 30-130, more preferably in the range of 40-120, even more preferably in the range of 40-110, and most preferably in the range of 40-100.

In further preferred embodiments of the present invention the second vegetable oil has an iodine value in the range of 30-120, more preferably in the range of 50-110, even more preferably in the range of 50-100, and most preferably in the range of 50-95.

In some preferred embodiments of the present invention the second vegetable oil has a peroxide value of at most 10 meq/kg, more preferably at most 5 meq/kg, even more preferably at most 3 meq/kg, and most preferably at most 2 meq/kg.

The peroxide value is determined according to Analysis E.

In some preferred embodiments of the present invention the second vegetable oil comprises a saturated vegetable oil and an unsaturated vegetable oil.

If the second oil contains several vegetable oils these may be added individually or may be premixed and added as the premix.

In the context of the present invention a “saturated vegetable oil” has an iodine value of less than 50 and an “unsaturated vegetable oil” has an iodine value of at least 50.

The saturated vegetable oil is preferably selected from the group consisting of palm stearin, palm kernel oil, shea stearin, coconut oil, hydrogenations of vegetable oil, fractions thereof, and/or a blend thereof.

The saturated vegetable oil is preferably selected form the group consisting of palm stearin, palm kernel oil, shea stearin, a hydrogenated vegetable oil or a combination thereof.

In some preferred embodiments of the present invention the second vegetable oil is a blend of different vegetable oils and/or oil fractions.

Preferably, the second vegetable oil comprises the saturated vegetable oil in an amount in the range of 1-80% w/w, more preferably 10-70% w/w, even more preferably 20-60% w/w, and most preferably 20-50% w/w.

Preferably, the second vegetable oil comprises the saturated vegetable oil in an amount in the range of 1-50% w/w, more preferably 2-40% w/w, even more preferably 4-30% w/w, and most preferably 4-25% w/w.

The saturated vegetable oil preferably has an iodine value in the range of 0-49, more preferably in the range of 10-45, even more preferably in the range of 14-42, and most preferably in the range of 20-40.

The second vegetable oil preferably comprises the unsaturated vegetable oil in an amount in the range of 20-99% w/w, more preferably 30-80% w/w, even more preferably 40-80% w/w, and most preferably 40-70% w/w.

In other preferred embodiments of the present invention the second vegetable oil comprises the unsaturated vegetable oil in an amount in the range of 20-100% w/w, more preferably 30-100% w/w, even more preferably 40-100% w/w, and most preferably 50-100% w/w.

The unsaturated vegetable oil preferably has an iodine value in the range of 50-130, more preferably in the range of 70-120, even more preferably in the range of 80-110, and most preferably in the range of 85-100.

In some preferred embodiments of the present invention the unsaturated vegetable oil is selected from the group consisting of Rapeseed oil, Sunflower oil, High oleic rapeseed oil, High oleic sunflower oil, Almond oil, avocado oil, Camelina oil, Corn oil, Cottonseed oil, Linseed oil, Olive oil, Palm oil, Palm olein, Peanut oil, Hazelnut oil, Mustard seed oil, Pumpkin seed oil, Rice bran oil, Safflower oil, Sesame oil, Soybean oil, Shea oil, Shea olein, Walnut oil, and a combination thereof.

More preferably the unsaturated vegetable oil is selected form the group consisting of high oleic sunflower oil, high oleic rapeseed oil, palm olein, shea olein, and a combination thereof.

Preferably, the method furthermore contains a step of filling the structurizing oil in a suitable container, such as e.g. drums, cans, buckets and/or a tanker truck.

Once filled, the structurizing oil is preferably kept at a fairly low temperature to reduce or avoid oxidation and/or degradation of the oil.

Yet an aspect of the invention pertains to a structurizing oil, preferably obtainable by the method described herein, containing
triglyceride in an amount in the range of 50-95% w/w,
diglyceride in an amount in the range of 2-40% w/w,
monoglyceride in an amount in the range of 0.2-8% w/w,
free fatty acids in an amount of at most 4% w/w,
glycerol in an amount of at most 1% w/w.

The present inventors have found that the interesterification changes the distribution of specific triglycerides, see e.g. Table 6 of Example 3, and this change is believed to contribute to the beneficial effects of the structurizing oil.

The present structurizing oil is preferably prepared from one or more vegetable oils and is therefore preferably of vegetable origin. Preferably, the structurizing oil does not contain fatty acids of non-vegetable origin.

In some preferred embodiments of the present invention the structurizing oil comprises a total amount of diglyceride in the range of 5-35% w/w, more preferably 10-32% w/w, even more preferably 12-30% w/w, and most preferably 15-30% w/w.

In other preferred embodiments of the present invention the structurizing oil comprises a total amount of diglyceride in the range of 2-20% w/w, more preferably 2-15% w/w, even more preferably 2-10% w/w, and most preferably 2-5% w/w.

In some preferred embodiments of the present invention the structurizing oil comprises a total amount of monoglyceride in the range of 0.4-8% w/w, more preferably 1.0-7% w/w, even more preferably 1.5-6% w/w, and most preferably 1.7-5% w/w.

In other preferred embodiments of the present invention the structurizing oil comprises a total amount of monoglyceride in the range of 0.2-7% w/w, more preferably 0.3-5% w/w, even more preferably 0.4-3% w/w, and most preferably 0.5-2% w/w.

In some preferred embodiments of the present invention the structurizing oil has a peroxide value of at most 10 meq/kg, more preferably at most 5 meq/kg, even more preferably at most 3 meq/kg, and most preferably at most 2 meq/kg.

In some preferred embodiments of the present invention the structurizing oil has an iodine value of at least 20, more preferably at least 40, even more preferably at least 50, and most preferably at least 60.

In some preferred embodiments of the present invention the structurizing oil has an iodine value in range of 40-100, even more preferably in the range of 40-90, and most preferably in the range of 50-90.

In some preferred embodiments of the present invention the structurizing oil has a content of glycidol equivalents of at most 10 ppm, more preferably at most 5 ppm, even more preferably at most 1 ppm, and most preferably at most 0.5 ppm.

The content of glycidol equivalents is determined according to Analysis C.

In some preferred embodiments of the present invention, the structurizing oil comprises water in an amount of at most 10% w/w, more preferably at most 5% w/w, even more preferably at most 1% w/w, and most preferably at most 0.5% w/w.

The structurizing oil preferably has a solid fat content (SFC) at the following temperatures of: 15 degrees C.: 0-25% w/w, more preferably 5-20% w/w, and most preferably 10-16% w/w, and 30 degrees C.: 0-12% w/w, more preferably 2-10% w/w, and most preferably 5-7% w/w.

A lower SFC makes it possible to process fat-containing food matrices such as e.g. margarines and frozen, aerated confections at lower temperatures and still control the fat structure.

In some preferred embodiments of the present invention, the structurizing oil, preferably obtainable by the method described herein, contains
triglyceride in an amount in the range of 70-85% w/w, and most preferably in the range of 75-85% w/w,
diglyceride in an amount in the range of 10-25% w/w, and most preferably in the range of 14-22% w/w
monoglyceride in an amount in the range of 0.5-4% w/w, and most preferably in the range of 1-3% w/w, and having:
a peroxide value of at most 5 meq/kg, and most preferably at most 2 meq/kg
an iodine value of 40-100, and most preferably 50-90, and
a content of glycidol equivalents of at most 5 mg/kg, and most preferably at most 1 mg/kg.

In other preferred embodiments of the present invention, the structurizing oil, preferably obtainable by the method described herein, contains
triglyceride in an amount in the range of 70-85% w/w, and most preferably in the range of 75-85% w/w,
diglyceride in an amount in the range of 10-25% w/w, and most preferably in the range of 14-22% w/w
monoglyceride in an amount in the range of 0.5-4% w/w, and most preferably in the range of 1-3% w/w
free fatty acids in an amount of at most 2% w/w, and most preferably at most 1% w/w,
glycerol in an amount of at most 1% w/w,
water in an amount of at most 1%, and most preferably at most 0.5% w/w, and having:
a peroxide value of at most 5 meq/kg, and most preferably at most 2 meq/kg
an iodine value of 40-100, and most preferably 50-90, and
a content of glycidol equivalents of at most 5 mg/kg, and most preferably at most 1 mg/kg.

In further preferred embodiments of the present invention, the structurizing oil, preferably obtainable by the method described herein, contains
triglyceride in an amount in the range of 70-85% w/w, and most preferably in the range of 75-85% w/w,
diglyceride in an amount in the range of 10-25% w/w, and most preferably in the range of 14-22% w/w
monoglyceride in an amount in the range of 0.5-4% w/w, and most preferably in the range of 1-3% w/w
free fatty acids in an amount of at most 2% w/w, and most preferably at most 1% w/w,
glycerol in an amount of at most 1% w/w,
water in an amount of at most 1%, and most preferably at most 0.5% w/w, and having:
a peroxide value of at most 5 meq/kg, and most preferably at most 2 meq/kg
an iodine value of 40-100, and most preferably 50-90,
a content of glycidol equivalents of at most 5 mg/kg, and most preferably at most 1 mg/kg, and
a solid fat content (SFC) at the following temperatures of:
15 degrees C.: 0-25% w/w, and most preferably 10-16% w/w, and
30 degrees C.: 0-12% w/w, and most preferably 2-10% w/w.

Another aspect of the invention pertains to the use of the present structurizing oil as a fat source in the production of a margarine type product.

The structurizing oil preferably contributes with at least 2% w/w of the total triglyceride of the margarine-type product, more preferably at least 5% w/w, even more preferably at least 15% w/w, and most preferably at least 25% w/w of the triglyceride of the margarine-type product.

In other preferred embodiments of the present invention, the structurizing oil contributes with at least 40% w/w of the total triglyceride of the margarine-type product, more preferably at least 70% w/w, even more preferably at least 80% w/w, and most preferably at least 90% w/w of the triglyceride of the margarine-type product. It may even be preferred that the structurizing oil provides at least 98% w/w of the triglyceride of the margarine-type product.

In some preferred embodiments of the present invention the structurizing oil contributes with 2-40% w/w of the total triglyceride of the margarine-type product, more preferably 5-30% w/w, even more preferably 10-25% w/w, and most preferably 10-20 w/w of the triglyceride of the margarine-type product.

In other preferred embodiments of the present invention the structurizing oil contributes with 2-25% w/w of the total triglyceride of the margarine-type product, more preferably 2-15% w/w, even more preferably 2-10% w/w, and most preferably 2-6 w/w of the triglyceride of the margarine-type product.

The oil product of the present invention surprisingly acts by structurizing margarine-type products, meaning that it appears to control the fat structure during the cooling process and particularly control of the viscosity and crystallisation rate. The structurizing functionality of the oil product is particularly advantageous in low fat margarine-type products.

The oil of the present invention surprisingly appears to provide the following benefits to the margarine-type product:
lowering the possible temperature of processing by controlling the solid fat content relative to the non-interesterified oil
smaller water droplet size which gives a more stable product and prevents water to be squeezed out,
smoother mouthfeel, and
a better resistance against microbial growth, due to the smaller water droplets.

The structurizing oil of the present invention furthermore surprisingly provides margarine-type products with an increased elasticity, which e.g. is a benefit for baked pastry applications.

The structurizing oil of the present invention furthermore surprisingly provides margarine-type products with an improved ability to incorporate sugar particles into the margarine during use. This is for example useful for cake applications and applications where sugar is whipped into margarine and provides more airy cakes (see e.g. Example 8).

Yet an aspect of the invention pertains to a method of producing a margarine-type product comprising the steps of
1) providing a fat phase comprising or even consisting of the structurizing oil as described herein or obtainable by a method as described herein,
2) providing an aqueous phase,
3) emulsifying the aqueous phase in the fat phase therefore by forming an emulsion
4) cooling the emulsion under shear to form a fat-crystallized water-in-oil emulsion,
5) packaging the fat-crystallized water-in-oil emulsion.

Step 5) may furthermore involve adding flavour and/or seasoning, and/or shaping the margarine prior to packaging.

The aqueous phase preferably comprises:
water in an amount of 5-95% w/w, more preferably 10-85% w/w, and most preferably 20-75% w/w,
salt in an amount of 0-6% w/w, more preferably 0-5% w/w, and most preferably 0-4% w/w,
Antioxidant in an amount of 0.01-5% w/w, more preferably 0.01-3% w/w, and most preferably 0.01-2% w/w,
flavors and vitamins in an amount of 0.01-5% w/w, more preferably 0.01-3% w/w, and most preferably 0.01-2% w/w.

It is often also preferred that the aqueous phase contains fiber, preferably in an amount of 0-30% w/w, more preferably 0-20% w/w, and most preferably 0-15% w/w.

In the context of the present invention the term “fiber” pertains to vegetable fibers which generally are based on arrangements of cellulose, often with lignin. Suitable examples include cotton, hemp, jute, flax, ramie, sisal, bagasse, citrus and banana.

The fat phase preferably comprises:
fat in an amount of 5-95% w/w, more preferably 10-85% w/w, and most preferably 20-75% w/w,
Antioxidant in an amount of 0.01-5% w/w, more preferably 0.01-3% w/w, and most preferably 0.01-2% w/w, and
Flavours and vitamins in an amount of 0.01-5% w/w, more preferably 0.01-3% w/w, and most preferably 0.01-2% w/w.

Alternatively but also preferred, the fat phase may comprise:
fat in an amount of 5-100% w/w, more preferably 60-100% w/w, and most preferably 80-100% w/w,
Antioxidant in an amount of 0-5% w/w, more preferably 0.01-3% w/w, and most preferably 0.01-2% w/w, and
Flavours and vitamins in an amount of 0-5% w/w, more preferably 0.01-3% w/w, and most preferably 0.01-2% w/w.

In some preferred embodiments of the present invention, the structurizing oil of the present invention is present in the fat phase in an amount sufficient to provide at least 2% w/w of the fat phase, more preferably at least 5% w/w of the fat phase, and most preferably at least 15% w/w of the fat phase.

In other preferred embodiments of the present invention, the structurizing oil of the present invention is present in the fat phase in an amount sufficient to provide at least 40% w/w of the fat phase, more preferably at least 70% w/w of the fat phase, and most preferably at least 90% w/w of the fat phase.

In further preferred embodiments of the present invention the structurizing oil of the present invention is present in the fat phase in an amount sufficient to provide 2-40% w/w of the fat phase, more preferably 5-30% w/w of the fat phase, and most preferably 10-25% w/w of the fat phase.

In other preferred embodiments of the present invention, the structurizing oil of the present invention is present in the fat phase in an amount sufficient to provide 2-25% w/w of the fat phase, more preferably 2-15% w/w of the fat phase, and most preferably 2-6% w/w of the fat phase.

In further preferred embodiments of the present invention the structurizing oil of the present invention is present in the fat phase in an amount sufficient to provide substantially all fat phase and is the only fat source of the margarine-type product.

In some preferred embodiments of the present invention the fat phase has a peroxide value of at most 10 meq/kg, more preferably at most 5 meq/kg, even more preferably at most 3 meq/kg, and most preferably at most 2 meq/kg.

In some preferred embodiments of the present invention the fat phase has an iodine value of at least 20, more preferably at least 40, even more preferably at least 50, and most preferably at least 60.

In some preferred embodiments of the present invention the fat phase has an iodine value in range of 40-100, even more preferably in the range of 40-90, and most preferably in the range of 50-90.

In some preferred embodiments of the present invention the fat phase has a content of glycidol equivalents of at most 10 ppm, more preferably at most 5 ppm, even more preferably at most 1 ppm, and most preferably at most 0.5 ppm.

The margarine-type product:
preferably contains the fat phase in an amount of about 10-about 90% w/w and the aqueous phase in an amount of about 10-about 90% w/w,
more preferably contains the fat phase in an amount of about 30-about 85% w/w and the aqueous phase in an amount of about 15-about 70% w/w,
most preferably contains the fat phase in an amount of about 40-about 80% w/w and the aqueous phase in an amount of about 20-about 60% w/w.

In some preferred embodiments of the present invention the fat phase comprises a total amount of diglyceride in the range of 2-20% w/w, more preferably 2-15% w/w, even more preferably 2-10% w/w, and most preferably 2-5% w/w.

In other preferred embodiments the fat phase comprises a total amount of monoglyceride in the range of 0.2-7% w/w, more preferably 0.3-5% w/w, even more preferably 0.4-3% w/w, and most preferably 0.5-2% w/w.

The margarine-type product may furthermore contain additional emulsifiers, such as e.g. isolates of monodiglycerides and/or lecithin, e.g. in a total amount of 0.01-5% w/w, more preferably 0.01-3% w/w, and most preferably 0.01-2% w/w. Such additional emulsifiers may be added to the fat phase and/or the aqueous phase in an amount of 0.01-5% w/w, more preferably 0.01-3% w/w, and most preferably 0.01-2% w/w.

However, in some preferred embodiments of the present invention no additional emulsifier is used for the preparation of the fat phase and the aqueous phase.

Yet an aspect of the invention pertains to a margarine-type product as described herein, preferably obtainable by the process described herein.

In the context of the present invention the term “margarine-type product” pertains to a fat-continuous emulsion of an aqueous phase in a fat phase. Margarine-type products, such as margarines, fat spreads, or butter blends with oil and fat content in emulsion from 10-90% can vary much in consistence from liquid to very solid depending on the fat composition. Such variations in consistency are based on variation of saturated, unsaturated and polyunsaturated fat composition. The fat types can variate from vegetable to animal fat and fish oil. The margarine, spreads and butter blends can have a very simple ingredients list with only water and emulsifying and/or crystalizing ingredients to more complex composition with salt, hydrocolloids, alginates, milk solids, milk proteins, vegetable proteins, fibers, starch, syrup, maltodextrins, sugar, fructose, glycose, gelatin, flavor, colour, antioxidants and vitamins.

The emulsification of the water and fat phase and the crystallization of the fat types is important for production of the margarine, spread and butter blends.

Preferably, the margarine type product comprises:
10-95% w/w fat
0-15 w/w Starch
0-17% w/w Fiber
0-5 w/w antioxidants, sugars, colours, flavours and/or vitamins
5-90% w/w water.

Another aspect of the invention pertains to the use of the present structurizing oil as an oil source in the production of a frozen, aerated confection.

The structurizing oil preferably contributes with at least 2% w/w of the total triglyceride of the frozen, aerated confection, more preferably at least 5% w/w, even more preferably at least 15% w/w, and most preferably at least 25% w/w of the triglyceride of the frozen, aerated confection.

In other preferred embodiments of the present invention, the structurizing oil contributes with at least 40% w/w of the total triglyceride of the frozen, aerated confection, more preferably at least 70% w/w, even more preferably at least 80% w/w, and most preferably at least 90% w/w of the triglyceride of the frozen, aerated confection. It may even be preferred that the structurizing oil provides at least 98% w/w of the triglyceride of the frozen, aerated confection.

In some preferred embodiments of the present invention the structurizing oil contributes with 2-40% w/w of the total triglyceride of the frozen, aerated confection, more preferably 5-30% w/w, even more preferably 10-25% w/w, and most preferably 10-20 w/w of the triglyceride of the frozen, aerated confection.

In other preferred embodiments of the present invention the structurizing oil contributes with 2-25% w/w of the total triglyceride of the frozen, aerated confection, more preferably 2-15% w/w, even more preferably 2-10% w/w, and most preferably 2-6 w/w of the triglyceride of the frozen, aerated confection.

The frozen, aerated confection is preferably an ice cream, ice milk, a mousse, or a frozen yoghurt.

The oil product of the present invention surprisingly acts by structurizing frozen, aerated confections, meaning that it appears to control the fat structure during the cooling process and particularly to control the viscosity and crystallisation rate. This is especially important in low fat frozen, aerated confections to avoid the formation of large ice crystals.

The structurizing oil surprisingly appears to provide the following benefits to the frozen, aerated confection:
Smoother texture by control of fat structure, i.e. body mouthfeel
Smaller water-ice crystals size, preventing a cold unpleasant mouthfeel
Improved heat shock resistance with less growth in water-ice crystal size, i.e. preventing a cold unpleasant mouthfeel which is especially critical when frozen confections are exposed to heat shock
Improved meltdown profile upon eating and prevents chewiness

A further aspect of the invention pertains to a method of producing a frozen, aerated confection comprising the steps of
1) providing a fat composition comprising or even consisting of the structurizing oil as described herein or obtainable by a method described herein,
2) providing an aqueous composition comprising at least sweetener and a protein,
3) emulsifying the fat composition in the aqueous composition therefore by forming a dessert emulsion,
4) optionally, resting the dessert emulsion at a temperature between 1-10 degrees C. to form a fat-crystallized dessert emulsion,
5) freezing the dessert emulsion to form the frozen, aerated confection.

The aqueous composition preferably comprises:
water in an amount of 20-95% w/w, more preferably 25-85% w/w, and most preferably 30-75% w/w,
sugars, syrups, sugar alcohols, sweeteners in a total amount of 0.1-30% w/w, more preferably 0.5-25% w/w, and most preferably 1-25% w/w,
milk solids non-fat and/or vegan non-fat solids in a total amount 0.1-30% w/w, more preferably 0.5-25% w/w, and most preferably 1-20% w/w,
stabilizers in an amount of 0.01-5% w/w, more preferably 0.01-4% w/w, and most preferably 0.01-3% w/w, and optionally
flavours and/or colours in an amount of 0-5% w/w, more preferably 0-4% w/w, and most preferably 0-3% w/w.

The fat composition preferably comprises:
a total amount of fat of 95-100% w/w, more preferably 96-100% w/w, and most preferably 97-100% w/w,
Flavors and colors in an amount of 0-5% w/w, more preferably 0-4% w/w, and most preferably 0-3% w/w.

The fat composition and the frozen, aerated confection preferably comprise saturated fatty acids in an amount of at least 40% w/w relative to total fatty acids, more preferably at least 50% w/w, and most preferably at least 60% w/w.

In some preferred embodiments of the present invention, the structurizing oil of the present invention is present in the fat composition in an amount sufficient to provide at least 2% w/w of the fat composition, more preferably at least 5% w/w of the fat composition, and most preferably at least 15% w/w of the fat composition.

In other preferred embodiments of the present invention, the structurizing oil of the present invention is present in the fat composition in an amount sufficient to provide at least 40% w/w of the fat composition, more preferably at least 70% w/w of the fat composition, and most preferably at least 90% w/w of the fat composition.

In further preferred embodiments of the present invention the structurizing oil of the present invention is present in the fat composition in an amount sufficient to provide 2-40% w/w of the fat composition, more preferably 5-30% w/w of the fat composition, and most preferably 10-25% w/w of the fat composition.

In other preferred embodiments of the present invention, the structurizing oil of the present invention is present in the fat composition in an amount sufficient to provide 2-25% w/w of the fat composition, more preferably 2-15% w/w of the fat composition, and most preferably 2-6% w/w of the fat composition.

In further preferred embodiments of the present invention the structurizing oil of the present invention is present in the fat composition in an amount sufficient to provide the entire fat composition and is the only fat source of the frozen, aerated confection.

In some preferred embodiments of the present invention the fat composition has a peroxide value of at most 10 meq/kg, more preferably at most 5 meq/kg, even more preferably at most 3 meq/kg, and most preferably at most 2 meq/kg.

In some preferred embodiments of the present invention the fat composition has an iodine value of at least 20, more preferably at least 40, even more preferably at least 50, and most preferably at least 60.

In some preferred embodiments of the present invention the fat composition has an iodine value in range of 40-100, even more preferably in the range of 40-90, and most preferably in the range of 50-90.

In some preferred embodiments of the present invention the fat composition has a content of glycidol equivalents of at most 10 ppm, more preferably at most 5 ppm, even more preferably at most 1 ppm, and most preferably at most 0.5 ppm.

The frozen, aerated confection:
preferably contains the fat composition in an amount of about 1 - about 30% w/w and the aqueous composition in an amount of about 70-about 99% w/w,
more preferably contains the fat composition in an amount of about 2 - about 80% w/w and the aqueous composition in an amount of about 20-about 98% w/w, and
most preferably contains the fat composition in an amount of about 5 - about 15% w/w and the aqueous composition in an amount of about 85-about 95% w/w.

In some preferred embodiments of the present invention the fat composition comprises a total amount of diglyceride in the range of 2-20% w/w, more preferably 2-15% w/w, even more preferably 2-10% w/w, and most preferably 2-5% w/w.

In other preferred embodiments the fat composition comprises a total amount of monoglyceride in the range of 0.2-7% w/w, more preferably 0.3-5% w/w, even more preferably 0.4-3% w/w, and most preferably 0.5-2% w/w.

The frozen, aerated confection may furthermore contain additional emulsifiers, such as e.g. isolates of monodiglycerides and/or lecithin, e.g. in a total amount of 0.01-5% w/w, more preferably 0.01-3% w/w, and most preferably 0.01-2% w/w. Such additional emulsifiers may be added to the fat phase and/or the aqueous phase in an amount of 0.01-5% w/w, more preferably 0.01-3% w/w, and most preferably 0.01-2% w/w.

However, in some preferred embodiments of the present invention no additional emulsifier is used for the preparation of the fat composition and the aqueous composition.

Yet an aspect of the invention pertains to a frozen, aerated confection obtainable by the present method. The frozen, aerated confection preferably comprises:
fat in an amount of 1-30% w/w, more preferably 2-20% w/w, and most preferably 5-15% w/w,
milk solids non-fat and/or vegan non-fat solids in a total amount of 1-20% w/w, more preferably 2-15% w/w, and most preferably 5-15% w/w,
optionally, sugars, syrups, sugar alcohols, and/or sweeteners in a total amount of 1-25% w/w, more preferably 2-20% w/w, and most preferably 4-15% w/w,
stabilizer in an amount of 0.01 -3% w/w, more preferably 0.1-3% w/w, and most preferably 0.5-3% w/w, and
water in an amount of 20-75% w/w, more preferably 30-70% w/w, and most preferably 40-70% w/w.

Useful examples of vegan non-fat solids are e.g. plant fibres and/or plant proteins, such as soy protein and/or pea protein.

In some preferred embodiments of the present invention the structurizing oil of the present invention is present in the frozen, aerated confection in an amount sufficient to provide 0.1-40% w/w of the fat of the frozen, aerated confection, more preferably 0.1-30% w/w of the fat of the frozen, aerated confection, and most preferably 0.1-25% w/w of the fat of the frozen, aerated confection.

In other preferred embodiments of the present invention, the structurizing oil of the present invention is present in the frozen, aerated confection in an amount sufficient to provide at least 40% w/w of the fat of the frozen, aerated confection, more preferably at least 70% w/w of the fat of the frozen, aerated confection, and most preferably at least 90% w/w of the fat of the frozen, aerated confection.

In further preferred embodiments of the present invention the structurizing oil of the present invention is present in the frozen, aerated confection in an amount sufficient to provide the entire fat composition and is the only fat source of the frozen, aerated confection.

In some preferred embodiments of the present invention the frozen, aerated confection is a vegan frozen, aerated confection and only contains vegan ingredients.

### EXAMPLES

### Methods of Analysis

### Analysis A: Gas Chromatography (GC) Analysis

### Analytical Procedure:

Each sample was dissolved in a pyridine/hexane mixture (50: 50) or in chloroform, derivatized with N-methyl-N-trimethylsilyl-trifluoroacetamide (MSTFA) and analysed on an Agilent 6890N gas chromatograph equipped with a FID detector and a split injection port. The column was a capillary J&W DB5 column and helium was used as carrier gas. Highly pure reference standards were used for establishing the correct calibration parameters for the analytes of interest.

Analysis B: Droplet Size Measurement with Low Field Nuclear Magnetic Resonance, Bruker Minispec mg20

### Standard Operating Procedure:

### Introduction:

The Gradient Strength Variation (G-Var) Droplet Size Analyzer with automation application determines in an automatic way the droplet size distribution in oil-in-water (0/W) emulsions (like mayonnaise, salad dressing or soft cheese) or in water-in-oil (W/O) emulsions (like margarine, low fat spread or butter) by Time Domain NMR. Tempering of the probe at 20 degrees C. (0/W) or at 5 degrees C. (W/O) is needed and can be achieved by external probe tempering with a thermostat/cryostat bath combination.

### System:

Minispec mq20 Oil/Water Droplet Size Analyzer.
Probe: PH H20-10-25-AVGX(Y).
Pulsed field gradient unit.
10 mm automation system.

### Tempering the Probe:

Temper the probe at 5 degrees C. (W/O) or at 20 degrees C. (O/W) using an external water/cryostat bath. The bath temperature must be adjusted in order to reach a constant temperature of 5 degrees C. (W/O) or 20 degrees C. (O/W) inside of the probe. Check the probe temperature with a thermometer. Make sure that no water condenses inside the probe and/or at the outside of the NMR tube. If some water condenses check the function of the nitrogen flow.

### Sample Preparation and Handling:

Fill the emulsion sample into the tube up to the recommended filling height: 1.5 cm. The sample is then handled according to the instruction in the Minispec mq20 software.

Preparation of the Calibration Sample Bruker supplies the calibration sample required for the G-Var method: CuSO4 solution in distilled water—the filling height is 1.5 cm. The CuSO4 solution should be stored in a cool dark place, e.g. in the refrigerator at 4 to 8 degrees C. Alternatively, the user can prepare their own calibration sample: ⋅0.5% CuSO4*5H2O aqueous solution, filling height 1.5 cm. Dissolve 0.5 g CuSO4*5H2O in 99.5 ml of distilled water. This sample has to be renewed every 3 months. Store it in a cool dark place, e.g. in the refrigerator at 4 to 8 degrees C.

### Analysis C: Determination of the Content of Glycidol Equivalents

The content of glycidol equivalents is determined according to AOCS Official Method Cd 29b-13 rev. 2017.

### Analysis D: Determination of the Amounts of Monoglyceride, Diglyceride, Triglyceride, Free Glycerol and Free Fatty Acids

The amounts of monoglyceride (MAG), diglyceride (DAG), triglyceride (TAG), free glycerol and free fatty acids (FFA) are determined according to AOCS Official Method Cd 11b-91, Reapproved 2009.

### Analysis E: Determination of Peroxide Value

The peroxide value of a sample is determined according to AOCS Official Method Cd 8b-90. The peroxide values are provided in the unit meq H202/kg sample.

### Analysis F: Determination of Iodine Value

The iodine value (IV) of a sample is determined according to AOAC Official Method 993.20. The iodine value is expressed as grams of iodine absorbed by the 100 g of the test portion following the specified procedure.

### Example 1

### Preparation of an Interesterified Oil A

Preparation of an interesterified oil based on a single oil without subsequent removal of free fatty acids.

### Materials and Methods:

High oleic sunflower oil (HOSO) 550g was added to a reaction flask with mechanical stirring and adjusted to the reaction temperature 40 degrees centigrade by controlled heating. Lipase Lipozyme TL (activity 33 KLU/g) 1.5 weight percentage of the oil was added and then reaction with continued mechanical stirring at the temperature 40 degrees centigrade. The reaction was ended by inactivation of the lipase activity by heating of the reaction blend to 100 degrees centigrade in not less than 30 minutes. The inactivated lipase was removed by filtration with celite as filtering aid.

### Results:

The degree of reaction was followed by GC-analysis, and as shown in Table 1 the interesterification was slowed down after 3-5 hours.

TABLE 1
The composition of mixture during the interesterification process.
Composition: weight percentage by GC-analysis vs. hours of reaction

| Time | MG | DG | TG | FFA | Free G |
|---|---|---|---|---|---|
| 0.0 | 0.5 | 4.9 | 92.8 | 1.8 | 0.0 |
| 1.0 | 2.4 | 15.0 | 73.3 | 9.2 | 0.1 |
| 2.0 | 2.9 | 17.5 | 69.1 | 10.4 | 0.1 |
| 3.0 | 3.1 | 18.1 | 68.0 | 10.8 | 0.1 |
| 4.0 | 3.1 | 18.4 | 67.4 | 10.9 | 0.1 |
| 5.0 | 3.2 | 18.8 | 66.9 | 11.1 | 0.1 |

### Conclusion:

The interesterified oil A based on a single oil HOSO and without removing of free fatty acid to a lower level was obtained after 5 hours of reaction with the composition: Monoglyceride (MG)=3.2% w/w, diglyceride (DG)=18.8% w/w, triglyceride (TG)=66.9% w/w, free fatty acids (FFA)=11.1% w/w and free glycerol (Free G)=0.1% w/w.

### Example 2

### Preparation of Structurizing Oil B

Preparation of structurizing oil B based on a single oil HOSO with removing of the free fatty acids to a lower level.

### Materials and Methods:

a) High oleic sunflower oil (HOSO) 77kg was added to a reaction plant with mechanical stirring and adjusted to the reaction temperature 40 degrees centigrade by controlled heating. Lipase Lipozyme TL (activity 11 KLU/g) 4.5 weight percentage of the oil was added and then reaction with continued mechanical stirring at the temperature 40 degrees centigrade. The reaction was ended by inactivation of the lipase activity by heating of the reaction blend to 100 degrees centigrade in not less than 30 minutes. The inactivated lipase was removed by filtration through 100 μm filter.

b) By distillation on a short patch distillation plant the free fatty acid was removed to a lower level of the product from a).

The removal of the free fatty acid to a lower level was done with use of a double column short path distillation plant, or by twice distillation on a single column short path distillation plant.

### Parameter Setting:

1. Distillation: Temperature=200 deg. centigrade and pressure=1000-1300 PA

2. Distillation: Temperature=200 deg. centigrade and pressure=400-450 PA

### Results:

The degree of reaction was followed by GC-analysis, and as shown in Table 2 the interesterification was slowed down after 3-5 hours. In table 3 the composition is compared before and after distillation.

TABLE 2
The composition of mixture during the interesterification process.
Composition: weight percentage by GC-analysis vs. hours of reaction

| Time | MG | DG | TG | FFA | Free G |
|---|---|---|---|---|---|
| 0.0 | 1.1 | 11.0 | 82.5 | 5.5 | 0.1 |
| 1.0 | 5.0 | 26.7 | 49.5 | 18.7 | 0.2 |
| 2.0 | 6.2 | 30.2 | 40.5 | 22.8 | 0.3 |
| 3.0 | 6.7 | 30.7 | 38.2 | 24.1 | 0.3 |
| 4.0 | 6.8 | 30.9 | 37.4 | 24.5 | 0.4 |
| 5.0 | 7.0 | 31.2 | 38.1 | 23.3 | 0.4 |
| 6.0 | 7.1 | 30.9 | 36.7 | 24.8 | 0.4 |

TABLE 3
The composition of the interesterified mixture
prior to, after 1. distillation and after 2. distillation
Composition: weight percentage by GC-analysis

| | MG | DG | TG | FFA | Free G |
|---|---|---|---|---|---|
| Feed | 7.3 | 26.2 | 42.3 | 23.8 | 0.4 |
| 1. distillation | 6.9 | 28.6 | 56.6 | 8.0 | 0.0 |
| 2. distillation | 4.0 | 29.7 | 65.7 | 0.6 | 0.0 |

### Conclusion:

Structurizing oil B based on a single oil HOSO and removing of free fatty acid to a lower level was obtained after 6 hours of reaction with the composition: Monoglyceride (MG)=4.0% w/w, diglyceride (DG)=29.7% w/w, triglyceride (TG)=65.7% w/w, free fatty acids (FFA)=0.6 w/w and free glycerol (Free G)=0.1% w/w.

### Example 3

### Preparation of Structurizing Oil C

Preparation of structurizing oil C based on a blend of oils HOSO and Palm stearin with removing of the free fatty acids to a lower level.

### Materials and Methods:

Example 3 was performed according to example 2, except a) using HOSO and Palm stearin (Palmotex 98T, AarhusKarlshamn) in a blend 60:40% w/w, b) using Lipase Lipozyme TL (activity 20 KLU/g) in 2.5 weight percentage of the oil, and c) Performed the reaction at temperature 50 degrees centigrade.

### Results:

The degree of reaction was followed by GC-analysis, and as shown in Table 4 the interesterification was slowed down after 2.5-5 hours. In Table 5 the composition is compared before and after distillation. The triglyceride distribution before, during and after the interesterification as well as after distillation is shown in Table 6.

TABLE 4
The composition of mixture during the interesterification process.
Composition: weight percentage by GC-analysis vs. hours of reaction

| Time | MG | DG | TG | FFA | Free G |
|---|---|---|---|---|---|
| 0.0 | 0.5 | 4.9 | 92.8 | 1.8 | 0.0 |
| 2.5 | 2.8 | 16.2 | 70.0 | 10.8 | 0.1 |
| 5.0 | 2.9 | 16.5 | 69.7 | 10.9 | 0.1 |

TABLE 5
The composition of the interesterified mixture
prior to, after 1. distillation and after 2. distillation
Composition: weight percentage by GC-analysis

| | MG | DG | TG | FFA | Free G |
|---|---|---|---|---|---|
| Feed | 2.9 | 16.5 | 69.7 | 10.9 | 0.1 |
| 1. distillation | 3.1 | 21.3 | 72.1 | 3.5 | 0.0 |
| 2. distillation | 1.7 | 17.9 | 79.8 | 0.6 | 0.0 |

TABLE 6
The triglyceride distribution in weight percentage
after reaction and distillation

| | Method DGF C-VI 14, GC-FID | | | |
|---|---|---|---|---|
| | Product: | | | |
| | | | | Example 3 |
| | Example 3 | Example 3 | Example 3 | after |
| Triglyceride: | t = 0 h | t = 2.5 h | t = 5 h | distillation |
| OOO | 25.1 | 29.3 | 31.4 | 31.5 |
| PLL | 0.9 | 0.9 | 0.9 | 0.9 |
| PLP | 4.1 | 2.3 | 2.6 | 2.6 |
| POO | 12.7 | 18.8 | 17.2 | 17.6 |
| POP | 20.0 | 13.5 | 14.2 | 14.3 |
| POS | 3.3 | 2.9 | 3.0 | 2.9 |
| PPP | 12.8 | 9.3 | 8.3 | 8.0 |
| PPS | 2.4 | 2.1 | 1.8 | 1.7 |
| SOS | 0.5 | 0.4 | 0.4 | 0.4 |
| MOP | 0.8 | 0.6 | 0.6 | 0.6 |
| PSS | 0.3 | 0.3 | 0.2 | 0.2 |
| SSS | 0.1 | 0.2 | 0.1 | 0.1 |
| OOS | 3.6 | 4.5 | 4.7 | 4.7 |
| PLO | 3.4 | 3.8 | 3.5 | 3.6 |
| OLO | 2.2 | 4.0 | 3.7 | 3.6 |
| SLL | 0.2 | 0.2 | 0.2 | 0.2 |
| LLO | 1.0 | 1.3 | 1.1 | 1.3 |
| LLL | 0.7 | 0.5 | 0.7 | 0.6 |
| PLS | 0.9 | 0.6 | 0.7 | 0.7 |
| SLO | 1.0 | 1.0 | 1.1 | 0.9 |
| Total | 96.0 | 96.5 | 96.4 | 96.4 |

L: Lauric acid;
P: Palmetic acid;
S: Stearic acid;
O: Oleic acid

### Structurizing Oil C Had:

an iodine value of 60.4 g I₂/100 g
a peroxide value of 0.8 meq O₂/kg
a glycidol content of 0.18 mg/kg
a solid fat content (SFC) of 33.0% w/w (0 degrees C.); 14.3% w/w (15 degrees C.); 4.73 w/w (30 degrees C.); 0% w/w (45 degrees C.), and
a water content of 0.2% w/w.

The solid fat content was measured according to IUPAC method 2.150(a).

The water content was measured according to the Karl Fischer method (ISO 8534:2017).

### Conclusion:

Structurizing oil C based on a blend of HOSO and Palm stearin (Palmotex 98T, AarhusKarlshamn) in a blend 3:2% w/w, and removing of free fatty acid to a lower level, was obtained after 5 hours of reaction and distillation with the composition: Monoglyceride (MG)=1.7% w/w, diglyceride (DG)=17.9% w/w, triglyceride (TG)=79.8% w/w, free fatty acids (FFA)=0.6 w/w and free glycerol (Free G)=0.0% w/w.

The triglyceride distribution has changed during the interesterification, where the biggest changes are among the triglycerides OOO, PLP, POO, POP, PPP, OOS and OLO, and then being almost unchanged during the distillation. The main triglycerides in the structurizing oil C is OOO=31.5% w/w, POO=17.6% w/w, POP=14.3% w/w, PPP=8.0% w/w and OOS=4.7% w/w.

### Example 4

### Margarine Based on Structurizing Oil B

Preparation of a 40% Margarine based on structurizing oil B, a single oil structurizing oil with a low level of FFA.

### Materials and Methods:

The water phase ingredients were mixed before adding to the fat phase and mixing. The structurizing oil B was added to the fat before mixing while in the comparing reference, without Structurizing oil B, monoglycerides E471 were added to the fat phase. After mixing of the water and fat phases a water-in-oil emulsion was formed. The emulsion was cooled and crystalized by using a Schroder margarine pilot plant VK60/400-4A2CV.

TABLE 7
Margarine recipes

| | | | 8.0% | | 6.0% | | 4.0% | |
|---|---|---|---|---|---|---|---|---|
| | | | Structurizing | | Structurizing | | Structurizing | |
| | Reference | | oil B | | oil B | | oil B | |
| | Trial A | | Trial B | | Trial C | | Trial D | |
| | % | kg | % | kg | % | kg | % | kg |
| Distilled monoglyceride, E471 | 0.50 | 0.18 | 0.00 | 0.00 | 0.00 | 0.00 | 0.00 | 0.00 |
| Structurizing oil B | 0.00 | 0.00 | 8.00 | 2.80 | 6.00 | 2.10 | 4.00 | 1.40 |
| Oil blend | | | | | | | | |
| Fritex 35, AarhusKarlshamn, | 5.71 | 2.00 | 5.71 | 2.00 | 5.71 | 2.00 | 5.71 | 2.00 |
| Sweden (Palm oil) | | | | | | | | |
| Pk4, Cargill, Germany | 12.29 | 4.30 | 12.29 | 4.30 | 12.29 | 4.30 | 12.29 | 4.30 |
| (interesterified blend of Palm | | | | | | | | |
| stearin and coconut oil) | | | | | | | | |
| Rapeseed oil | 21.50 | 7.53 | 14.00 | 4.90 | 16.00 | 5.60 | 18.00 | 6.30 |
| Total oil phase | 40.00 | 14.00 | 40.00 | 14.00 | 40.00 | 14.00 | 40.00 | 14.00 |
| Water phase | | | | | | | | |
| Water | 59.18 | 20.71 | 59.18 | 20.71 | 59.18 | 20.71 | 59.18 | 20.71 |
| Salt | 0.70 | 0.25 | 0.70 | 0.25 | 0.70 | 0.25 | 0.70 | 0.25 |
| Potassium sorbate | 0.10 | 0.04 | 0.10 | 0.04 | 0.10 | 0.04 | 0.10 | 0.04 |
| Colour annatto | 0.02 | 0.01 | 0.02 | 0.01 | 0.02 | 0.01 | 0.02 | 0.01 |
| Total water phase | 60.00 | 21.00 | 60.00 | 21.00 | 60.00 | 21.00 | 60.00 | 21.00 |
| Total recipe | 100.00 | 35.00 | 100.00 | 35.00 | 100.00 | 35.00 | 100.00 | 35.00 |

Test 1. The water droplet size in the prepared margarines was measured by low field NMR with use of Bruker Minispec mq20, where the droplet size distribution was measured relative to the droplet volume. In table 8 the result of the droplet size measurements is shown.

TABLE 8
Droplet size measurement with low field Nuclear
Magnetic Resonance, Bruker Minispec mq20

| | Droplet size in microns volume | | |
|---|---|---|---|
| | distribution, each sample measured twice: | | |
| | 2.5% of | 50% of | 97.5% of |
| Trial | droplet volume | droplet volume | droplet volume |
| A | 2.42 | 6.17 | 15.87 |
| B | 1.54 | 5.27 | 18.07 |
| C | 2.09 | 10.03 | 48.33 |
| D | 1.59 | 16.16 | 164.63 |

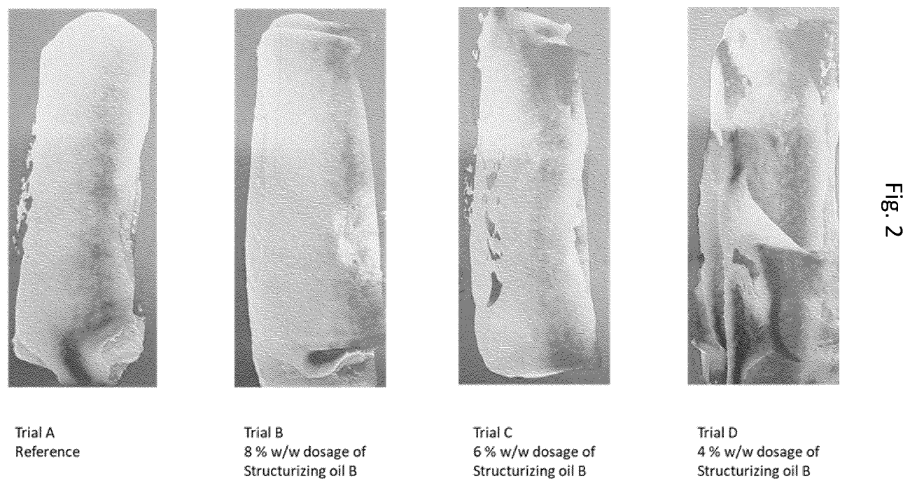

Test 2. The stability of the prepared margarines was compared by squeezing them out on a surface, and by looking on how good the water was bounded in the structure of fat. The test was performed by swiping a pallet knife back and forth four times while pressing down or squeezing the margarine to the surface. If clear water was squeezed out the emulsion was unstable. In FIG. 2 it is shown that margarines from Trial B and C have similar stability as the reference margarine from Trial A. In Trial D the margarine was unstable with interruptions and clear water at the surface after squeezing.

### Conclusion:

Margarine 40% based on structurizing oil B showing comparable performance to the reference

Margarine with use of monoglyceride E471. The margarines were compared in droplet size distribution, and in stability when squeezed out on a surface.

In Trial B, where the structurizing oil B was used in a dosage of 8% w/w, the margarine has a water droplet size distribution where 2.5% of droplet volume have a droplet size of 1.54 microns or less, 50% of the droplet volume have a droplet size of 5.27 microns or less and 97.5% of the droplet volume have a droplet size of 18.07 microns or less.

In the squeeze test the margarines were stable with a dosage of at least 6.0% of structurizing oil B where at lower dosage water was squeezed out.

### Example 5

### Margarine Based on Structurizing Oil C

Preparation of a 40% Margarine based on structurizing oil C, a blend of oils structurizing oil with a low level of FFA.

### Materials and Methods:

The water phase ingredients were mixed before adding to the fat phase and mixing. The structurizing oil C was added to the fat before mixing while in the comparing reference, without structurizing oil C, monoglycerides E471 were added to the fat phase. After mixing of the water and fat phases a water-in-oil emulsion was formed. The emulsion was cooled and crystalized by using a Schrôder margarine pilot plant VK60/400-4A2CV.

TABLE 9
Margarine recipes

| | | | 18.83% | | 14.12% | | 9.40% | |
|---|---|---|---|---|---|---|---|---|
| | | | Structurizing | | Structurizing | | Structurizing | |
| | Reference | | oil C | | oil C | | oil C | |
| | Trial A | | Trial B | | Trial C | | Trial D | |
| | % | kg | % | kg | % | kg | % | kg |
| Distilled monoglyceride, E471 | 0.50 | 0.18 | 0.00 | 0.00 | 0.00 | 0.00 | 0.00 | 0.00 |
| Structurizing oil C | 0.00 | 0.00 | 18.83 | 6.59 | 14.12 | 4.94 | 9.40 | 3.29 |
| Oil blend | | | | | | | | |
| Fritex 35, AarhusKarlshamn, | 5.71 | 2.00 | 1.37 | 0.48 | 2.46 | 0.86 | 3.54 | 1.24 |
| Sweden (Palm oil) | | | | | | | | |
| Pk4, Cargill, Germany | 12.29 | 4.30 | 12.29 | 4.30 | 12.29 | 4.30 | 12.29 | 4.30 |
| (interesterified blend of Palm | | | | | | | | |
| stearin and coconut oil) | | | | | | | | |
| Rapeseed oil | 21.50 | 7.53 | 7.51 | 2.63 | 11.14 | 3.90 | 14.77 | 5.17 |
| Total oil phase | 40.00 | 14.00 | 40.00 | 14.00 | 40.00 | 14.00 | 40.00 | 14.00 |
| Water phase | | | | | | | | |
| Water | 59.18 | 20.71 | 59.18 | 20.71 | 59.18 | 20.71 | 59.18 | 20.71 |
| Salt | 0.70 | 0.25 | 0.70 | 0.25 | 0.70 | 0.25 | 0.70 | 0.25 |
| Potassium sorbate | 0.10 | 0.04 | 0.10 | 0.04 | 0.10 | 0.04 | 0.10 | 0.04 |
| Colour annatto | 0.02 | 0.01 | 0.02 | 0.01 | 0.02 | 0.01 | 0.02 | 0.01 |
| Total water phase | 60.00 | 21.00 | 60.00 | 21.00 | 60.00 | 21.00 | 60.00 | 21.00 |
| Total recipe | 100.00 | 35.00 | 100.00 | 35.00 | 100.00 | 35.00 | 100.00 | 35.00 |

### Results:

Test 1. The water droplet size in the prepared margarines was measured by low field NMR with use of Bruker Minispec mq20, where the droplet size distribution was measured relative to the droplet volume. In table 10 the result of the droplet size measurements is shown.

TABLE 10
Droplet size measurement with low field Nuclear
Magnetic Resonance, Bruker Minispec mq20

| | Droplet size in microns volume | | |
|---|---|---|---|
| | distribution, each sample measured twice: | | |
| | 2.5% of | 50% of | 97.5% of |
| Trial | droplet volume | droplet volume | droplet volume |
| A | 2.01 | 3.52 | 6.16 |
| B | 0.97 | 3.15 | 10.36 |
| C | 3.28 | 3.29 | 3.30 |
| D | 1.28 | 4.15 | 13.57 |

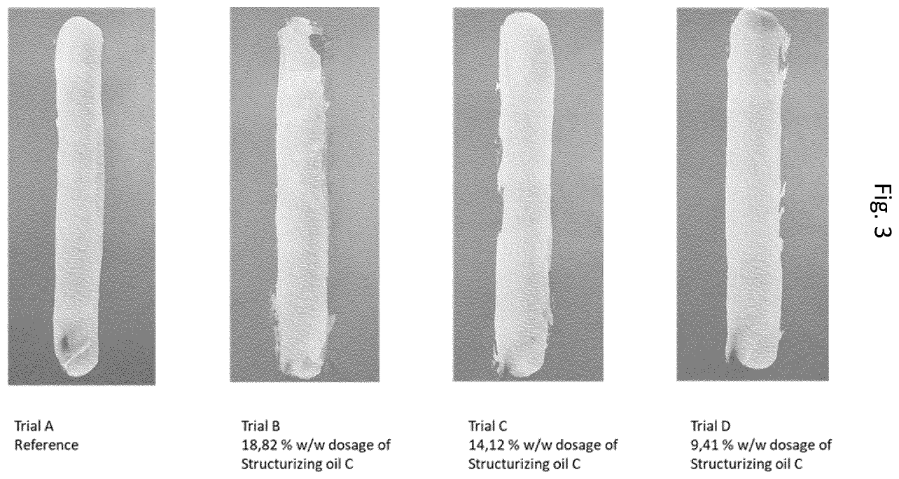

Test 2. The stability of the prepared margarines was compared by squeezing them out on a surface, and by looking on how good the water is bounded in the structure of fat. The test was performed by swiping a pallet knife back and forth four times while pressing down or squeezing the margarine to the surface. If clear water was squeezed out the emulsion was unstable. In FIG. 3 it is shown that margarines from Trial B, C and D has similar stability as the reference margarine from Trial A. In all the performed trials the margarines were stable without interruptions and no clear water at the surface after squeezing.

### Conclusion:

Margarine 40% based on structurizing oil C is showing comparable or better performance to the reference Margarine with use of monoglyceride E471. The margarines were compared in droplet size distribution, and in stability when squeezed out on a surface.

In Trial C, where the structurizing oil C was used in a dosage of 14.12% w/w, the margarines have a water droplet size distribution where 2.5% of droplet volume have a droplet size of 3.28 microns or less, 50% of the droplet volume have a droplet size of 3.29 microns or less and 9.75 of the droplet volume have a droplet size of 3.3 microns or less.

In the squeeze test the margarines were stable with a dosage of at least 9.4% of structurizing oil C.

### Example 6

### Ice Cream Based on Structurizing Oil B

Preparation of a 10% Ice cream based on structurizing oil B, a single oil structurizing oil with low level FFA.

### Materials and Methods:

The water phase ingredients were mixed before adding to the fat phase and mixing. The structurizing oil B was added to the fat before mixing while in the comparing reference, without Structurizing oil B, monodiglycerides E471 were added to the water phase in a mixture with stabilizers Locust bean gum and Guar gum.

After mixing of the water and fat phases the mix was pasteurized, sterilized, homogenized, cooled and aged overnight at 0-5 degrees centigrade but not lower.

The ice cream was obtained by the use of an ice-cream mixer and cooler, Gram Equipment GIF 400.

TABLE 11
Ice cream recipe and mixing

| | Reference | | Example 6 | |
|---|---|---|---|---|
| Ingredients | % | g | % | g |
| Fat-phase: | | | | |
| Structurizing oil B | | | 3.00 | 660 |
| Crystal Special | 10.00 | 2200 | 7.00 | 1540 |
| Water-phase: | | | | |
| Mono- and diglycerides of | 0.50 | 110 | — | — |
| fatty acids E 471: 70% w/w | | | | |
| Locust bean gum E 410: 15% w/w | | | | |
| Guar gum E 412: 15% w/w | | | | |
| Locust bean gum E 410: 50% w/w | — | — | 0.20 | 44 |
| Guar gum E 412: 50% w/w | | | | |
| Skimmed Milk Powder (SMP) | 11.40 | 2508 | 11.40 | 2508 |
| Sugar | 11.50 | 2530 | 11.50 | 2530 |
| Glucose Syrup. 39DE Powder | 2.50 | 550 | 2.50 | 550 |
| Vanilla | 0.10 | 22 | 0.10 | 22 |
| Water | 64.00 | 14080 | 64.30 | 14146 |
| Total | 100.00 | 20000 | 100.00 | 22000 |
| Mixing | | | | |

| Homogenization. | 75 degrees C./140 bar | |
|---|---|---|
| Pasteurization. | 85 degrees C./5 sec (NZS) | |
| Cooling to. | <5 degrees C., aged over night | |
| Mix appearance | OK | OK |
| Mix viscosity. BfDVIII. | 280 | 270 |
| 20 rpm. sp. 2 | | |

The recipes and mixing parameters as well as the mixing performance before freezing are shown in Table 11 where the freezing parameters and the results are shown in Table 12.

TABLE 12
Ice cream freezing and results

| Gram Equipment. GIF 400 | Reference | Example 6 |
|---|---|---|
| Mix flow. l/h | 50 | 50 |
| Overrun. % | 100 | 100 |
| Dasher speed. rpm | 300 | 300 |
| Viscosity. % | 45 | 45 |
| Cylinder pressure. bar | 4 | 4 |
| Outlet temp. degrees C. | −6.8 | −6.5 |
| ½ liter weight. g | 284/278 | 282/272 |
| Evaluation, ice cream | Mouthfeel with | Mouthfeel with |
| | good meltdown | good meltdown |
| | without water- | without water- |
| | ice crystals. | ice crystals. |
| | No shrinkage. | No shrinkage |
| Evaluation, ice cream | Mouthfeel with | Mouthfeel with |
| after heat shock | good meltdown | good meltdown |
| | with few water- | with few water- |
| | ice crystals. | ice crystals. |
| | Little to no | Little to no |
| | shrinkage. | shrinkage. |

### Conclusion:

Ice cream based on structurizing oil B showing comparable performance to the reference ice cream with use of monodiglyceride E471. The ice creams was compared in ½ liter weight, both measured twice, and evaluated in mouthfeel before and after heat shock. The mixes before freezing were also compared in viscosity and appearance. Only small differences when comparing the results of the performed trials were seen.

### Example 7

### Ice Cream Based on Structurizing Oil C

Preparation of a 10% Ice cream based on structurizing oil C, a blend of oils structurizing oil with low level FFA.

### Materials and Methods:

The water phase ingredients were mixed before adding to the fat phase and mixing. The structurizing oil C was added to the fat before mixing while in the comparing reference, without Structurizing oil C, monodiglycerides E471 were added to the water phase in a mixture with stabilizers Locust bean gum and Guar gum.

After mixing of the water and fat phases the mix was pasteurized, sterilized, homogenized, cooled and aged overnight at 0-5 degrees centigrade but not lower.

The ice cream was obtained by the use of an ice-cream mixer and cooler, Gram Equipment GIF 400.

TABLE 13
Ice cream recipe and mixing.

| | Reference | | Example 7 | |
|---|---|---|---|---|
| Ingredients | % | g | % | g |
| Fat-phase: | | | | |
| Structurizing oil C | | | 6.00 | 1320 |
| Crystal Special | 10.00 | 2200 | 4.00 | 880 |
| Water-phase: | | | | |
| Mono- and diglycerides of fatty | 0.50 | 110 | — | — |
| acids E 471: 70% w/w | | | | |
| Locust bean gum E 410: 15% w/w | | | | |
| Guar gum E 412: 15% w/w | | | | |
| Locust bean gum E 410: 50% w/w | — | — | 0.20 | 44 |
| Guar gum E 412: 50% w/w | | | | |
| Skimmed Milk Powder (SMP) | 11.40 | 2508 | 11.40 | 2508 |
| Sugar | 11.50 | 2530 | 11.50 | 2530 |
| Glucose Syrup. 39DE Powder | 2.50 | 550 | 2.50 | 550 |
| Vanilla | 0.10 | 22 | 0.10 | 22 |
| Water | 64.00 | 14080 | 64.30 | 14146 |
| Total | 100.00 | 20000 | 100.00 | 22000 |
| Mixing | | | | |

| Homogenization. | 75 degrees C./140 bar | |
|---|---|---|
| Pasteurization. | 85 degrees C./5 sec (NZS) | |
| Cooling to. | <5 degrees C. and aged over night | |
| Mix appearance | OK | OK |
| Mix viscosity. BfDVIII. | 270 | 232 |
| 20 rpm. sp. 2 | | |

### Results:

The recipes and mixing parameters as well as the mixing performance before freezing are shown in Table 13 where the freezing parameters and results are shown in Table 14.

TABLE 14
Ice cream freezing and results.

| Gram Equipment. GIF 400 | Reference | Example 7 |
|---|---|---|
| Mix flow. l/h | 50 | 50 |
| Overrun. % | 100 | 100 |
| Dasher speed. rpm | 325 | 325 |
| Viscosity. % | 45 | 45 |
| Cylinder pressure. bar | 4.5 | 4.5 |
| Outlet temp. degrees C. | −6.7 | −6.4 |
| ½ liter weight. g | 282/279 | 278/291 |
| Evaluation, ice cream | Mouthfeel with | Mouthfeel with |
| | good meltdown | good meltdown |
| | without water- | without water- |
| | ice crystals. | ice crystals. |
| | No shrinkage. | No shrinkage. |
| Evaluation, ice cream | Mouthfeel with | Mouthfeel with |
| after heat shock | good meltdown | good meltdown |
| | with few water- | with few water- |
| | ice crystals. | ice crystals. |
| | Little to no | Little to no |
| | shrinkage. | shrinkage. |

### Conclusion:

Ice cream based on structurizing oil C showing comparable performance to the reference ice cream with use of monodiglyceride E471. The ice creams were compared in ½ liter weight, both measured twice, and evaluated in mouthfeel before and after heat shock. The mixes before freezing were also compared in viscosity and appearance. Only small differences when comparing the results of the performed trials were seen.

### Example 8

### Cake and Cream Margarine Based on Structurizing Oil C

Preparation of a Cake and Cream Margarine based on structurizing oil C of Example 3.

### Materials and Methods:

The water phase ingredients were mixed before adding to the fat phase and mixing. The structurizing oil C was added to the fat before mixing while in the comparing reference, without structurizing oil C, Palsgaard® 1388, E471 and E475, were added to the fat phase. After mixing of the water and fat phases a water-in-oil emulsion was formed. The emulsion was cooled and crystallized by using a Schroder margarine pilot plant VK60/400-4A2CV.

TABLE 14
Cake and Cream Margarine recipes

| | | | 15.00% | | 10.00% | | 7.50% | |
|---|---|---|---|---|---|---|---|---|
| | | | Structurizing | | Structurizing | | Structurizing | |
| | Reference | | oil C | | oil C | | oil C | |
| | Trial A | | Trial B | | Trial C | | Trial D | |
| | % | kg | % | kg | % | kg | % | kg |
| Palsgaard ® 1388 (Blend | 0.80 | 0.28 | 0.00 | 0.00 | 0.00 | 0.00 | 0.00 | 0.00 |
| of mono diglycerides, | | | | | | | | |
| E471 and polyglycerol | | | | | | | | |
| ester, E475) | | | | | | | | |
| Lecithin, E322 | 0.20 | 0.07 | 0.00 | 0.00 | 0.00 | 0.00 | 0.00 | 0.00 |
| Structurizing oil C | 0.00 | 0.00 | 15.00 | 5.25 | 10.00 | 3.50 | 7.50 | 2.63 |
| Oil blend | | | | | | | | |
| Fritex 35, AarhusKarlshamn, | 19.00 | 6.65 | 13.50 | 4.72 | 15.50 | 5.42 | 16.50 | 5.77 |
| Sweden (Palm oil) | | | | | | | | |
| Pk4, Cargill, Germany | 31.00 | 10.85 | 31.00 | 10.85 | 31.00 | 10.85 | 31.00 | 10.85 |
| (interesterified blend of Palm | | | | | | | | |
| stearin and coconut oil) | | | | | | | | |
| Rapeseed oil | 21.00 | 7.35 | 12.50 | 4.38 | 15.50 | 5.43 | 17.00 | 5.95 |
| Coconut oil | 8.00 | 2.80 | 8.00 | 2.80 | 8.00 | 2.80 | 8.00 | 2.80 |
| Total oil phase | 80.00 | 28.00 | 40.00 | 28.00 | 80.00 | 28.00 | 80.00 | 28.00 |
| Water phase | | | | | | | | |
| Water | 19.00 | 6.65 | 19.00 | 6.65 | 19.00 | 6.65 | 19.00 | 6.65 |
| Salt | 1.00 | 0.35 | 1.00 | 0.35 | 1.00 | 0.35 | 1.00 | 0.35 |
| Colour annatto | 0.01 | 0.004 | 0.01 | 0.004 | 0.01 | 0.004 | 0.01 | 0.004 |
| Total water phase | 20.01 | 7.004 | 20.01 | 7.004 | 20.01 | 7.004 | 20.01 | 7.004 |
| Total recipe | 100.01 | 35.004 | 100.00 | 35.004 | 100.00 | 35.004 | 100.00 | 35.004 |

### Test 1. All-in-One Cake.

The margarines obtained from Trial A-D of Example 8 were evaluated in an All-in-one cake model based on the following recipe and method.

Recipe: 400 g Cake margarine, 400 g Powdered sugar, 400 g eggs, 440 g reform flour and 5 g Baking powder.

Method: All ingredients are mixed in a Hobart mixer with spatula for 1 min. in speed 1 and 5 min. in speed 2. Weigh in 350 g into a baking tin then baking for 45-50 min. at 180 degree Celsius.

### Test 2. Whipping Cream.

The margarines obtained from Trial A-D of Example 8 were furthermore evaluated in a whipping cream model based on the following recipe and method.

Recipe: 500 g Cake margarine and 350 g Powdered sugar

Method: The ingredients are mixed in a Hobart mixer with whisker for 1 min. at speed 1, scape down, and then at speed 2, and measure overrun every 5 min.

Results:

TABLE 15
Raw dough density (50 ml tin), cake volume (BVM6630 with software
Volcalc version 3,4,4,113 from Perten Instruments) and softness
measurements (TA.XT plus Texture Analyser with software Exponent
version 6,1,14,0 from Stable Micro Systems, the lower number
in gram (g) the higher softness), each sample measured twice.

| | Raw dough | Cake | Cake |
|---|---|---|---|
| Trial | density (g/L) | volume (ml) | softness (g) |
| A | 812 | 807 | 711 |
| B | 789 | 790 | 649 |
| C | 796 | 809 | 688 |
| D | 799 | 789 | 688 |

In table 16 the results of the measurement of overrun are shown.

TABLE 16
Whipping overrun measurements (100 ml
tin), each sample measured twice.

Density of Whipping (g/L)

| Trial | 5 min. | 10 min. | 15 min. | 20 min |
|---|---|---|---|---|
| A | 561 | 433 | 437 | 465 |
| B | 533 | 425 | 432 | 473 |
| C | 549 | 404 | 411 | 467 |
| D | 583 | 442 | 455 | 424 |

### Conclusion:

Cake and cream margarines based on structurizing oil C surprisingly had a comparable or better performance relative to the reference Margarine with use of Palsgaard® 1388, blend of mono-and diglycerides, E471 and polyglycerol ester, E475, and lecithin. The margarines were compared in all-in-one cake and in whipping cream.

In all the trials where the structurizing oil C were used (Trials B-D) the all-in-one cake had a lower raw dough density (characteristic of a more airy dough), a comparable cake volume and a better cake softness, compared with the reference (Trial A).

In all the trials where the structurizing oil C was used (Trials B-D) the whipping cream had comparable or better overrun performance, measured as density every 5 minutes of whipping, compared with the reference trial (Trial A).

The emulsifier system used in Trial A represented the state of the art emulsifier system for Cake and cream Margarines and it was surprising that it could be replaced by the use of structurizing oil C.

### Example 9

### Pastry Margarine Based on Structurizing Oil C

Preparation of a Pastry Margarine based on structurizing oil C of Example 3.

### Materials and Methods:

The water phase ingredients were mixed before adding to the fat phase and mixing. The structurizing oil C was added to the fat before mixing while in the reference (Trial A) containing Palsgaard® 1302, E471 and E475, was added to the fat phase. After mixing of the water and fat phases a water-in-oil emulsion was formed. The emulsion was cooled and crystallized by using a Schrôder margarine pilot plant VK60/400-4A2CV.

TABLE 17
Pastry Margarine recipes

| | | | 15.00% | | 10.00% | | 7.50% | |
|---|---|---|---|---|---|---|---|---|
| | | | Structurizing | | Structurizing | | Structurizing | |
| | Reference | | oil C | | oil C | | oil C | |
| | Trial A | | Trial B | | Trial C | | Trial D | |
| | % | kg | % | kg | % | kg | % | kg |
| Palsgaard ® 1302 (Blend | 0.80 | 0.80 | 0.00 | 0.00 | 0.00 | 0.00 | 0.00 | 0.00 |
| of mono- and diglycerides, | | | | | | | | |
| E471 and polyglycerol | | | | | | | | |
| ester, E475) | | | | | | | | |
| Lecithin, E322 | 0.50 | 0.50 | 0.00 | 0.00 | 0.00 | 0.00 | 0.00 | 0.00 |
| Structurizing oil C | 0.00 | 0.00 | 15.00 | 12.00 | 10.00 | 8.00 | 7.50 | 6.00 |
| Oil blend | | | | | | | | |
| Fritex 35, AarhusKarlshamn, | 35.50 | 35.50 | 30.80 | 24.64 | 32.80 | 26.24 | 33.80 | 27.04 |
| Sweden (Palm oil) | | | | | | | | |
| Pk4, Cargill, Germany | 31.20 | 31.20 | 31.20 | 24.96 | 31.20 | 24.96 | 31.20 | 24.96 |
| (interesterified blend of Palm | | | | | | | | |
| stearin and coconut oil) | | | | | | | | |
| Rapeseed oil | 12.00 | 12.00 | 3.00 | 2.40 | 6.00 | 4.80 | 7.50 | 6.00 |
| Total oil phase | 80.00 | 80.00 | 80.00 | 64.00 | 80.00 | 64.00 | 80.00 | 64.00 |
| Water phase | | | | | | | | |
| Water | 19.00 | 19.00 | 19.00 | 15.20 | 19.00 | 15.20 | 19.00 | 15.20 |
| Salt | 1.00 | 1.00 | 1.00 | 0.80 | 1.00 | 0.80 | 1.00 | 0.80 |
| Colour annatto | 0.01 | 0.01 | 0.01 | 0.008 | 0.01 | 0.008 | 0.01 | 0.008 |
| Total water phase | 20.01 | 20.01 | 20.01 | 16.008 | 20.01 | 16.008 | 20.01 | 16.008 |
| Total recipe | 100.01 | 100.01 | 100.01 | 80.008 | 100.01 | 80.008 | 100.01 | 80.008 |

### Puff Pastry Test.

The puff pastry test was performed with the following recipe and method.

Recipe: 100 g Pastry margarine, 1000 g flour, 10 g salt and 550 g water. Roll in with another 900 g of Pastry margarine.

### Method:

1. Preparing the dough: Add all ingredients, except the roll in pastry margarine, to the mixer (KEMPER type: SPL). Knead the dough: 2 min. low speed and 2 min. high speed.

2. Roll dough and margarine one half turn and one double turn (1×3 and 1×4) with a thickness of 10 mm in 6 steps before folding. After the first 2 turns let the paste rest for 20 min. in the refrigerator with a thickness at 15 mm.

3. After the resting time proceed according to step 1 (1×3 and 1×4). Finish at a thickness of 15 mm. The paste is then cut into 2 pieces and rests for 20 min. in the refrigerator. Making up the puff pastry squares: The paste is double over (total layers 288) and pinned down to a thickness of 4.5 mm. The squares are then made by cutting around the stencil.

4. Resting: After making, the squares rest for 90 min. at 20° C. before baking.

5. Baking: Oven temperature=215° C. (approx.); Baking time=15 min.

6. After baking the pastry squares should stand for 2 hours before evaluation.

The dough samples were evaluated with respect to dough performance (numbers of layers and plasticity) and the baking performance (height, width, expansion, and crumb/layers) are shown.

### Results:

In table 18 the results of the dough performance (numbers of layers and plasticity) and the baking performance (height, width, expansion, and crumb/layers) are shown.

TABLE 18
Dough and Baking performance

| | Trial A | Trial B | Trial C | Trial D |
|---|---|---|---|---|
| Rolled dough | | | | |
| Layers | 288 | 288 | 288 | 288 |
| Plasticity | good | good | good | good |
| Baked Squares | | | | |
| Height (mm) | 495 | 515 | 425 | 430 |
| Width (mm) | 620 | 620 | 620 | 620 |
| Expansion (times) | 11.0 | 11.4 | 9.4 | 9.6 |
| Crumb/layers | good | good | good | good |

### Conclusion:

Pastry Margarine based on structurizing oil C surprisingly showed similar or better performance relative to the reference Margarine with use of Palsgaard® 1302, blend of mono- and diglycerides, E471 and polyglycerol ester, E475, and lecithin. The margarines were compared in a Puff Pastry test.

In all the trials, where the structurizing oil C was used, the Puff Pastry dough had the right numbers of layers and a good plasticity and the baked squares had bigger, similar, or lower heights and similar widths. The Trial B-margarine provided the highest expansion after baking (11.4 times expansion). The Trial-C margarine resulted in an expansion of 9.4 times and the Trial D Margarine in an expansion of 9.6 times. The Trial A margarine (reference) provided an expansion of 11.0 times.

In all the trials, where the structurizing oil C was used, the baked squares had a good crumb and a number of layers which was similar to the reference trial.

The emulsifier system used in Trial A represented the state of the art emulsifier system for pastry margarines and it was surprising that it could be replaced by the use of structurizing oil C.

1. A method of producing a structurizing oil comprising:
a) subjecting a mixture comprising a first vegetable oil, water, and enzyme having lipase activity to interesterification,
b) subjecting the interesterified mixture of step a) to:
i) reduction of the lipase activity,
ii) reduction of the content of free fatty acids (ffa) selectively relative to the partial glycerides,
iii) optionally, reduction of the water content,
wherein steps i), ii), and iii) may be performed in any order, thereby providing a ffa-reduced mixture,
c) optionally, mixing the ffa-reduced mixture of step b) with a second vegetable oil to obtain an oil blend,
wherein the ffa-reduced mixture of step b) and/or the oil blend of step c) is recovered as the structurizing oil.

2. The method according to claim 1 wherein the mixture comprises the first vegetable oil in an amount in the range of 10-99.9% w/w, more preferably 30-99.8% w/w, even more preferably 50-99.7% w/w, and most preferably 60-99.7% w/w.

3. The method according to any of the preceding claims wherein the first vegetable oil has an iodine value in range of 20-100, even more preferably in the range of 40-90, and most preferably in the range of 50-90.

4. The method according to any of the preceding claims wherein the first vegetable oil comprises a saturated vegetable oil and an unsaturated vegetable oil.

5. The method according to any of the preceding claims wherein the mixture comprises a total amount of triglyceride of an amount in the range of 50-99.9% w/w, more preferably 60-99% w/w, even more preferably 70-97% w/w, and most preferably 75-95% w/w.

6. The method according to any of the preceding claims wherein the mixture comprises water in an amount in the range of 0.001-5% w/w, more preferably 0.01-2% w/w, even more preferably 0.05-1% w/w, and most preferably 0.1-0.5% w/w.

7. The method according to any of the preceding claims wherein step b) is performed in one of the following the sequences:
i) followed by ii),
i) followed by ii) followed by iii),
i) followed by iii) followed by ii),
ii) followed by i),
ii) followed by i) followed by iii),
ii) followed by iii) followed by i),
iii) followed by i) followed by ii), or
iii) followed by ii) followed by i)

8. The method according to any of the preceding claims wherein step b.i) involves inactivating the enzyme, preferably heating to a temperature in the range of 80 to 250 degrees C., and more preferably 85 -120 degrees C., and most preferably 90-100 degrees C.

9. The method according to claim 8 wherein step b.i) furthermore involves removing the inactivated enzyme by filtration or centrifugation, preferably by filtration.

10. The method according to any of the preceding claims wherein step b.i) involves removing the solid phase holding the enzyme from the interesterified mixture.

11. The method according to any of the preceding claims wherein step b.ii) reduces the content of ffa of the interesterified mixture to at most 51% w/w relative to the weight of the interesterified mixture, more preferably at most 3% w/w, even more preferably at most 2% w/w, and most preferably at most 1% w/w.

12. The method according to any of the preceding claims wherein step b.ii) involves or even consists of distillation and/or extraction, most preferably distillation.

13. The method according to any of the preceding claims comprising step b.iii).

14. The method according to claim 13 wherein the water content of the interesterified mixture is reduced to at most 1% w/w, more preferably at most 0.5% w/w, even more preferably at most 0.1% w/w, and most preferably at most 0.01% w/w.

15. The method according to any of the preceding claims, comprising step c) and wherein the oil blend of step c) comprises the structurizing oil in an amount of at least 2% w/w.

16. The method according to any of the preceding claims wherein the method furthermore contains a step of filling the structurizing oil in a suitable container, such as e.g. a drums, cans, buckets and/or tanker truck.

17. A structurizing oil, preferably obtainable by one or more of claims 1-16, containing
triglyceride in an amount in the range of 50-95% w/w,
diglyceride in an amount in the range of 2-40% w/w,
monoglyceride in an amount in the range of 0.2-6% w/w,
free fatty acids in an amount of at most 4% w/w,
glycerol in an amount of at most 1% w/w.

18. The structurizing oil according to claim 17, wherein the structurizing oil has a peroxide value of at most 10 meq/kg, more preferably at most 5 meq/kg, even more preferably at most 3 meq/kg, and most preferably at most 2 meq/kg.

19. The structurizing oil according to claim 17 or 18 wherein the structurizing oil has an iodine value of at least 20, more preferably at least 40, even more preferably at least 50, and most preferably at least 60.

20. The structurizing oil according to any of claims 17-19 having an iodine value in range of 40-100, even more preferably in the range of 40-90, and most preferably in the range of 50-90.

21. The structurizing oil according to any of claims 17-20 having a content of glycidol equivalents of at most 10 ppm, more preferably at most 5 ppm, even more preferably at most 1 ppm, and most preferably at most 0.5 ppm.

22. A method of producing a margarine-type product comprising the steps of
1) providing a fat phase comprising or even consisting of the structurizing oil according to one or more of claims 17-21 or obtainable by a method according to one or more of claims 1-16
2) providing an aqueous phase,
3) emulsifying the aqueous phase in the fat phase therefore by forming an emulsion
4) cooling the emulsion under shear to form a fat-crystallized water-in-oil emulsion,
5) packaging the fat-crystallized water-in-oil emulsion.

23. The method according to claim 22 wherein the structurizing oil contributes with at least 2% w/w of the total triglyceride of the margarine-type product, more preferably at least 5% w/w, even more preferably at least 15% w/w, and most preferably at least 25% w/w of all of the triglyceride of the margarine-type product.

24. The margarine type product obtainable by claim 22 or 23.

25. A method of producing a frozen, aerated confection comprising the steps of
1) providing a fat composition comprising or even consisting of the structurizing oil according to one or more of claims 17-21 or obtainable by a method according to one or more of claims 1-16,
2) providing an aqueous composition comprising at least a sweetener and a protein,
3) emulsifying the fat composition in the aqueous composition therefore by forming a dessert emulsion
4) optionally, resting the dessert emulsion at a temperature between 1-10 degrees C. to form a fat-crystallized dessert emulsion,
5) freezing the dessert emulsion to form the frozen, aerated confection.

26. The method according to claim 25 wherein the structurizing oil contributes with at least 2% w/w of the total triglyceride of the frozen aerated confection, more preferably at least 5% w/w, even more preferably at least 15% w/w, and most preferably at least 25% w/w of all of the triglyceride of the frozen aerated confection.

27. The frozen, aerated confection obtainable by claim 25 or 26.

28. Use of the structurizing oil according to any of claims 17-21 or obtainable by a method according to one or more of claims 1-16, as ingredient, for the production of a margarine-type product or for the production of a frozen, aerated confection, preferably wherein the structurizing oil contributes with at least 2% w/w of the total triglyceride of the margarine or the frozen aerated confection, more preferably at least 5% w/w, even more preferably at least 15% w/w, and most preferably at least 25% w/w of all of the triglyceride of the margarine or the frozen aerated confection.
